(19) United States
(12) Patent Application Publication
(10) Pub. No.: US 2016/0362353 A1
(43) Pub. Date: Dec. 15, 2016

(54) INTEGRATED PROCESS FOR PRODUCING CELLULOSIC PULP AND POLYOLS STREAM

(71) Applicant: BIOCHEMTEX S.P.A, Tortona (IT)

(72) Inventors: Marco BERNARDI, Blogna (IT); Patrizia FERRANDO, Lu (IT); Srinivasan RAGHAVENDRAN, Wilmington, NC (US); Andrea BONANNI, Tortona (IT); Alessandra FRATTINI, Castelnuovo Scrivia (IT); Dario GIORDANO, Tortona (IT)

(73) Assignee: Biochemtex S.p.A., Tortona (IT)

(21) Appl. No.: 15/121,085

(22) PCT Filed: Feb. 13, 2015
(86) PCT No.: PCT/EP2015/053062

§ 371 Date: Aug. 24, 2016

Related U.S. Application Data
(60) Provisional application No. 61/943,757, filed on Feb. 24, 2014.

(30) Foreign Application Priority Data
Apr. 14, 2014 (IT) MI2014A 000698

(51) Int. Cl. C07C 29/132; D21C 3/02; D21C 3/04; C07C 29/60; D21C 3/10; D21C 3/12; D21C 3/14; D21C 11/00; D21C 3/06; D21C 3/08

(52) U.S. Cl. CPC C07C 29/132; D21C 3/06; D21C 3/02; D21C 3/04; D21C 3/08; D21C 3/10; D21C 3/12; D21C 3/14; D21C 11/00; C07C 29/60

(57) ABSTRACT

It is disclosed an integrated process for producing at least a cellulosic pulp comprising cellulose in the form of fibers and a polyols stream from a ligno-cellulosic feedstock comprising cellulose, hemicellulose and lignin. The process comprises the steps of:

a) Treating the ligno-cellulosic feedstock to produce the cellulosic pulp and at least a liquid sugar stream comprising water and monomeric sugars derived from the hemicellulose of the ligno-cellulosic feedstock;

b) Catalytically converting the monomeric sugars of the liquid sugar stream to a polyols mixture, comprising primary polyols and secondary polyols;

c) Separating at least a portion of the polyols mixture into at least the polyols stream and a residual stream, wherein the polyols stream comprises the majority by weight of the primary polyols and the residual stream comprises the majority by weight of the secondary polyols;

d) Recovering a first thermal energy from the residual stream in a first recovery unit.

### FIELD OF THE INVENTION

The present invention relates to an integrated process for producing cellulosic pulp and polyols stream

### BACKGROUND

In the pulping industry, different processes are used for extracting a cellulosic pulp from ligno-cellulosic feedstocks, which are typically softwoods and hardwoods. Even if also mechanical treatments may be used, most diffused processes comprise a cooking treatment of the ligno-cellulosic feedstock with chemicals for solubilizing the most portion of the lignin and the hemicellulose, thereby producing a cellulosic fiber suspension in liquid solution called “brown stock”. By means of washing treatments, the brown stock is separated in a stream comprising the cellulosic fibers and one or more effluent streams, comprising the spent cooking chemicals and the solubilized lignin and hemicellulose. The clean pulp (stock) can be bleached in the bleach plant or left unbleached, depending on the end use. In the dissolving pulp process, which produces high purity cellulose, at least a portion of the hemicellulose is pre-extracted from the ligno-cellulosic feedstock by means of a hydro-thermal pre-treatment prior to treating the ligno-cellulosic feedstock depleted of the extracted hemicellulose with chemicals.

A common feature to all the processes in the pulp industry is the fact that they are very high energy demanding. The effluent streams are usually sent to a recovery boiler, where they are burnt generating heat from the solubilized lignin and hemicellulose. Heat is usually used to produce steam which is used in the process and in making electricity, similar to a conventional steam power plant. In the recovery boiler, chemicals are also recovered from the effluent stream and recycled in the process. As heat is a low value product, the conventional pulping processes only take a partial advantage from the ligno-cellulosic feedstock. Considering that hemicellulose has a low heating value, about half of that of lignin, burning hemicellulose to produce heat is not a convenient strategy for hemicellulose valorization. Moreover, the effluent streams, coming for the washing steps, are diluted and they must be subjected to many evaporation steps to increase the dry matter content before being burnt, at the expenses of the energy balance.

In the framework of biorefinery concept, different integrated processes for converting hemicellulose to various value-added products, such as alcohols, carboxylic acids and many others have been proposed. As an example, in the process described in Mao H. et al., “Technical economic evaluation of a hardwood biorefinery using the “near-neutral” hemicellulose pre-extraction process”, J. Biobased Mater. Bioenergy 2(2) p. 177-185 (2008), a portion of the hemicellulose is extracted from wood prior to pulping and converted into acetic acid and ethanol while using the extracted wood chips to produce Kraft pulp. In the paper, an existing Kraft pulp mill was considered as the base case. The pulp production was maintained constant and the hemicellulose extraction process was added to the fiber line. The hemicellulose extraction process occurs in a separated impregnation vessel prior to the continuous digester for pulp production. The extraction is carried out using green liquor (mostly Na2CO3+Na2S). The process disclosed for hemicellulose extraction and conversion to ethanol and acetic acid includes wood extraction for hemicellulose removal, flashing of the extract to produce preheating steam, recycling a portion of the extract back to the extraction vessel for the purpose of raising the solids content of the extract, acid hydrolysis using sulfuric acid for conversion of the oligomeric carbohydrates into monomeric sugars and cleavage of lignin-carbohydrate covalent bonds, filtration to remove precipitated lignin, liquid-liquid extraction followed by distillation to remove acetic acid and furfural from the sugar solution, liming to raise the pH to that required for fermentation, fermentation of five and six-carbon sugars and glucoronic acid to ethanol and finally distillation and upgrading the product to pure ethanol.

In Lyytikainen et al., “The change of hemicellulose extraction on fiber charge properties and retention behaviour of kraft pulp fibers” (2011), BioResources 6(1), p. 219-231, it is proposed a next generation technology for an integrated forest biorefinery, wherein the hemicellulose is extracted by means of an alkaline treatment from bleached kraft pulp.

The conversion process of the extracted streams comprising hemicellulose produces one or more residual streams which are usually sent to a waste water treatment facility.

On the other hand, the valorization of the hemicellulose to produce chemical end-products instead of thermal energy reduces the heating value of the effluent stream. There is thereby the need to integrate the heating value of the effluent stream which has been depleted from hemicellulose.

### BRIEF DESCRIPTION OF THE INVENTION

It is disclosed an integrated process for producing at least a cellulosic pulp comprising cellulose in the form of fibers and a polyols stream from a ligno-cellulosic feedstock comprising cellulose, hemicellulose and lignin. The process comprises the steps of:

a) Treating the ligno-cellulosic feedstock to produce the cellulosic pulp and at least a liquid sugar stream comprising water and monomeric sugars derived from the hemicellulose of the ligno-cellulosic feedstock;

b) Catalytically converting the monomeric sugars of the liquid sugar stream to a polyols mixture, comprising primary polyols and secondary polyols;

c) Separating at least a portion of the polyols mixture into at least the polyols stream and a residual stream, wherein the polyols stream comprises the majority by weight of the primary polyols and the residual stream comprises the majority by weight of the secondary polyols;

d) Recovering a first thermal energy from the residual stream in a first recovery unit.

It is also disclosed that the treatment of the ligno-cellulosic feedstock may comprise the steps of:
i. socking the ligno-cellulosic feedstock in the presence of a process fluid comprising water at a temperature between 100° C. and 210° C. for a time between 1 minute and 24 hours to produce a soaking liquid comprising oligomeric sugars and a soaked ligno-cellulosic feedstock;
ii. treating the soaking liquid comprising oligomeric sugars to produce at least the liquid stream comprising monomeric sugars;
iii. treating the soaked ligno-cellulosic feedstock in the presence of a chemical agent to produce at least a cellulosic stream comprising the cellulosic pulp and an effluent stream comprising at least a portion of the lignin and at least a portion of the chemical agent or a derivative of the chemical agent;
iv. recovering the cellulosic pulp, and
v. recovering from the effluent stream a recycled chemical agent comprising the chemical agent or the derivative of the chemical agent and a second thermal energy in a second recovery unit.

It is further disclosed that the process fluid may further comprise a portion of the chemical agent or a portion of the recycled chemical agent.

It is also disclosed that the chemical agent may comprise at least a compound selected from the group consisting of a sulfite, a bisulfite, sodium hydroxide and sodium carbonate.

It is further disclosed that the sulfite may comprise a counter ion which is selected from the group consisting of sodium ion, calcium ion, potassium ion, magnesium ion, and ammonium ion.

It is also disclosed that the bisulfite may comprise a counter ion which is selected from the group consisting of sodium ion, calcium ion, potassium ion, magnesium ion, and ammonium ion.

It is further disclosed that the catalytic conversion may comprise the steps of:

a) Hydrogenating the liquid sugar stream by contacting the liquid sugar stream with a hydrogenation catalyst in the presence of Hydrogen, at a hydrogenation pressure in the range of 30 bar to 150 bar and at a hydrogenation temperature in the range of 50° C. to 200° C., and for a hydrogenation time sufficient to produce a hydrogenated mixture comprising water and at least a sugar alcohol;

b) Conducting hydrogenolysis of at least a portion of the hydrogenated mixture, by contacting the at least a portion of the hydrogenated mixture with a hydrogenolysis catalyst in the presence of OH— ions and Hydrogen, at a hydrogenolysis pressure in the range of 40 bar to 170 bar, at a hydrogenolysis temperature and for a hydrogenolysis time sufficient to produce the polyols mixture.

It is also disclosed that the primary polyols may comprise at least a compound selected from the group consisting of ethylene glycol, propylene glycol, and mixture thereof.

It is further disclosed that the secondary polyols may comprise at least a compound selected from the group consisting of glycerol, arabitol, threitol, erythrithol and an unreacted sugar alcohol, and mixture thereof.

It is also disclosed that the residual stream may further comprise a compound selected from the list consisting of lactic acid, formic acid, and glycolaldehyde.

It is further disclosed that the residual stream may have a percent dry matter by weight which is a value greater than 80, 85, 90, 93, 95, and 98.

It is also disclosed that the residual stream may be inserted in the first recovery unit at a temperature which is greater than a value selected from the group consisting of 60° C., 70° C., and 80° C.

It is further disclosed that the first recovery unit and the second recovery unit may be the same recovery unit.

It is also disclosed that at least a portion of the residual stream and at least a portion of the effluent stream may be combined together before being inserted in the recovery unit.

It is further disclosed that the recovery unit may comprise a recovery boiler.

It is also disclosed that at least a portion of the first thermal energy and/or at least a portion of the second thermal energy may be used in the integrated process.

It is further disclosed that at least a portion of the first thermal energy and/or at least a portion of the second thermal energy may be converted to electrical energy.

### BRIEF DESCRIPTION OF FIGURES

FIG. 1 is a schematic representation of the invention.

FIG. 2 is schematic representation of another embodiment of the invention.

### DETAILED DESCRIPTION OF THE INVENTION

It is disclosed an integrated process for converting a ligno-cellulosic feedstock to value-added products obtained from the hemicellulose of the ligno-cellulosic feedstock and to a cellulosic pulp. Specifically, the value-added products are produced in the form of a polyols stream.

Polyols are compounds with multiple hydroxyl functional groups available for organic reactions. A molecule with two hydroxyl groups is a diol, one with three is a triol, one with four is a tetrol and so on.

Monomeric polyols such as glycerin, pentaerythritol, ethylene glycol and sucrose often serve as the starting point for polymeric polyols, which are generally used to produce other polymers. Polymeric polyols are usually polyethers or polyesters. Common polyether diols are for instance polyethylene glycol and polypropylene glycol.

Another class of polymeric polyols is the polyesters. Polyesters are formed by condensation or step-growth polymerization of diols and dicarboxylic acids (or their derivatives), for example diethylene glycol reacting with phthalic acid. Alternatively, the hydroxyl group and the carboxylic acid (or their derivatives) may be within the same molecule.

The ligno-cellulosic feedstock is treated to extract at least a portion of the hemicellulose from the ligno-cellulosic feedstock in soluble form, for producing at least a liquid stream comprising monomeric sugars derived from the hemicellulose of the ligno-cellulosic feedstock. The ligno-cellulosic feedstock which has been depleted from at least a portion of the hemicellulose is then converted to a cellulosic pulp, which is the well-known product of the pulp industry. The liquid stream comprising monomeric sugars is subjected to a catalytic conversion, which converts at least a portion of the monomeric sugars to a polyols mixture. In the context of the present disclosure, the polyols mixture comprises primary polyols and secondary polyols, being a primary polyol a polyol which is a desired product of the conversion process of the ligno-cellulosic feedstock, or which may be used to produce the desired product. A secondary polyols is a polyol which is not a desired product and in general it represents a by-product. The polyols mixture may further comprise compounds which are not polyols, such as for instance unreacted monomeric sugars, intermediated compounds or unwanted compounds.

The catalytic conversion preferably comprises an hydrogenation reaction of monomeric sugars and may comprise one or more reactions steps.

In the case of a single step conversion, the liquid sugar stream is contacted with a catalyst at suitable conditions to convert at least a portion of the monomeric sugars of the liquid sugar stream to the polyols mixture comprising primary polyols and secondary polyols. Reaction conditions may comprise for instance a reaction temperature in a range suitable for promoting the conversion. The reaction may occur in the presence of a gas, preferably hydrogen, at a reaction pressure in suitable ranges for promoting the conversion reaction.

In the case of a multi-steps conversion, the steps are preferably conducted sequentially. For instance, in a sequential two-step conversion, the liquid sugar stream is contacted with a first catalyst at first reaction conditions suitable to convert at least a portion of the monomeric sugars of the liquid sugar stream to an intermediate product or products. First reaction conditions may comprise for instance a first reaction temperature in a suitable range for promoting the conversion of at least a portion of the monomeric sugars to the intermediate product or products. The reaction may occur in the presence of a first gas, preferably hydrogen, at a first reaction pressure in suitable ranges for promoting the conversion reaction. of the monomeric sugars to the intermediate product or products. The intermediate product or products are then contacted with a second catalyst at second reaction conditions suitable to convert at least a portion of intermediate product or products to the polyols mixture. Second reaction conditions may comprise for instance a second reaction temperature in a suitable range for promoting the conversion of at least a portion of the intermediate product or products to the polyols mixture. The reaction may occur in the presence of a second gas, preferably hydrogen, at a second reaction pressure in a suitable range for promoting the conversion of at least a portion of the intermediate product or products to the polyols mixture. The first catalyst and the second catalyst may be the same catalyst.

The two steps may take place in two different reactors, and the intermediate product or products are preferably removed from the first reactor and inserted in the second reactor. The intermediate product or products may be further subjected to one or more conditioning steps, such as for instance filtration, purification, separation, before being inserted in the second reactor.

The catalytic reaction may be conducted in batch or continuous mode.

The polyols mixture is then separated into at least two streams, the polyols stream and a residual stream. Even if it is desirable to separate completely the primary polyols and the secondary polyols, thereby the polyols stream comprising the primary polyols and the residual stream comprising the secondary polyols, the separation step may be not able to obtain a complete separation of primary polyols and secondary polyols. The polyols stream may comprise the majority by weight of the total amount of the primary polyols of the polyols mixture, preferably more than 50% by weight of the total amount of the primary polyols, more preferably more than 60%, even more preferably more than 70%, even yet more preferably more than 80%, most preferably more than 90%. The residual stream may comprise the majority by weight of the total amount of the secondary polyols, preferably more than 50% by weight of the total amount of the secondary polyols of the polyols mixture, more preferably more than 60%, even more preferably more than 70%, even yet more preferably more than 80%, most preferably more than 90%. The polyols stream may further comprise a portion of the secondary polyols, as well as the residual stream may further comprise a portion of the primary polyols.

In one embodiment, the polyols mixture is first separated in more than two intermediate streams, none of which streams comprises the majority by weight of the total amount of the primary polyols and the majority weight of the total amount of the secondary polyols. In this case, the intermediate streams may be further processed to produce the polyols stream and the residual stream, for instance by combining the intermediate streams in a suitable way to obtain the polyols stream and the residual stream.

As the residual stream has a remarkable total heating value, and may be concentrated, that is it may contain a low amount of water, inventors have found that thermal energy may be conveniently recovered from it, thereby supplying at least a portion of the energy, more specifically of the thermal energy, needed to conduct the disclosed process.

Thereby, in the disclosed process, the residual stream is used to produce thermal energy in a first recovery unit. In this way, it is obtained the further advantage of reducing the amount of streams comprising organic compounds sent to a waste water treatment.

The disclosed process is an integrated process for converting the ligno-cellulosic feedstock to at least two different products, the cellulosic pulp and the polyols stream, wherein means for producing each product are preferably co-located in the same site. A further advantage of the disclosed process is that some equipments may be used both in the cellulosic pulp and the polyols stream production steps.

In some preferred embodiments, the disclosed process is implemented as a retrofit of an existing plant for producing cellulosic pulp, wherein equipments needed for producing the polyols stream are added to the existing plant and previously existing equipments are adapted for receiving and processing one or more streams from some steps of production of the polyols stream.

Treatments to Produce Cellulosic Pulp

The cellulosic pulp is a fibrous material prepared by chemically or mechanically separating cellulose fibers of the ligno-cellulosic feedstock. The cellulosic pulp comprises mainly cellulose in the form of fibers, even if it may comprise also bundles of fibers, which are groups of fibers which have not been separated by the process. Apart from cellulose, which is the main component, cellulosic pulp may still comprise a portion of the lignin and a portion of the hemicellulose of the ligno-cellulosic feedstock, which eventually have been modified by the treatment used to produce the pulp.

The cellulosic pulp may be prepared by any process used in the pulp industry known in the art and still to be invented, provided that it extracts or can be modified to extract at least a portion of the hemicellulose from the ligno-cellulosic feedstock.

In an embodiment, the process used to produce the cellulosic pulp comprises a mechanical pulp treatment. Manufactured grindstones with embedded silicon carbide or aluminum oxide can be used to grind small wood logs called “bolts” to make stone pulp. If the wood is steamed prior to grinding it is known as pressure ground wood pulp. Most modern mills use chips rather than logs and ridged metal discs called refiner plates instead of grindstones. If the chips are just ground up with the plates, the pulp is called refiner mechanical pulp. Steam treatment significantly reduces the total energy needed to make the pulp and decreases the damage (cutting) to fibres. Mechanical pulps are used for products that require less strength, such as newsprint and paperboards.

In another embodiment, the process used to produce the cellulosic pulp comprises a thermo-mechanical pulp treatment. Thermo-mechanical pulp is pulp produced by processing the ligno-cellulosic feedstock, preferably in the form of chips, by using heat and a mechanical refining movement. It is a two stage process where the logs are first stripped of their bark and converted into small chips. These chips have usually a moisture content of around 25-30% and a mechanical force is applied to the wood chips in a crushing or grinding action which generates heat and water vapor and softens the lignin thus separating the individual fibres. The pulp is then screened and cleaned, any clumps of fibres are reprocessed. This process gives a high yield of fiber from the timber (around 95%) and as the lignin has not been removed, the fibres are hard and rigid.

In another embodiment, the process used to produce the cellulosic pulp comprises a chemi-thermo-mechanical pulp treatment. Wood chips can be pretreated with sodium carbonate, sodium hydroxide, sodium sulfite and other chemicals prior to refining with equipments similar to a mechanical mill. The conditions of the chemical treatment are much less vigorous (lower temperature, shorter time, less extreme pH) than in a chemical pulping process since the goal is to make the fibres easier to refine, not to remove lignin as in a fully chemical process.

In another embodiment, the process used to produce the cellulosic pulp comprises a chemical pulp treatment. Chemical pulp is produced by combining the ligno-cellulosic feedstock in form of chips and chemicals in large vessels known as digesters where heat and the chemicals break down the lignin, which binds the cellulose fibres together, without seriously degrading the cellulose fibres. Chemical pulp is used for materials that need to be stronger or combined with mechanical pulps to give a product different characteristics. The kraft process is the dominant chemical pulping method, with sulfite process being second. Historically soda pulping was the first successful chemical pulping method. After several hours in the digester, the chips or cut plant material breaks down into a slurry of thick consistency and is typically “blown” or squeezed from the outlet of the digester through an airlock. The sudden change in pressure results in a rapid expansion of the fibers, separating the fibres even more. The resulting fiber suspension in water solution is called “brown stock”. Brown stock washers, using countercurrent flow, remove the spent cooking chemicals and degraded lignin and hemicellulose. The extracted liquid, known as black liquor in the kraft process, and red or brown liquor in the sulfite processes, is concentrated, burned and the sodium and sulfur compounds recycled in the recovery process. Lignosulfonates are a useful byproduct recovered from the spent liquor in the sulfite process. The clean pulp (stock) can be bleached in the bleach plant or left unbleached, depending on the end use. The stock is sprayed onto the pulp machine wire, water drains off, more water is removed by pressing the sheet of fibers, and the sheet is then dried. The obtained sheets of pulp are several millimeters thick and have a coarse surface and may be further processed to produce different kind of products.

In a preferred embodiment, the chemical pulp treatment used to produce the cellulosic pulp comprises a kraft process, which may be modified to increase the removal of the hemicellulose from the ligno-cellulosic feedstock. Without limiting the way to implement the kraft process within the scope of the present disclosure, the kraft process may be described as follows. The kraft process comprises a treatment of wood chips with a mixture of sodium hydroxide and sodium sulfide, known as white liquor, that breaks the bonds that link lignin to the cellulose. The ligno-cellulosic feedstock in the form of chips normally first enter the pre-steaming where they are wetted and preheated with steam. Cavities inside fresh wood chips are partly filled with liquid and partly with air. The steam treatment causes the air to expand and about 25% of the air to be expelled from the chips. The next step is to impregnate the chips with black and white liquor. Air remaining in chips at the beginning of liquor impregnation is trapped within the chips. The impregnation can be done before or after the chips enters the digester and is normally done below 100° C. The cooking liquors consist of a mixture of white liquor, water in chips, condensed steam and weak black liquor. In the impregnation, cooking liquor penetrates into the capillary structure of the chips and low temperature chemical reactions with the wood begin. A good impregnation is important to get a homogeneous cook and low rejects. About 40-60% of all alkali consumption in the continuous process occurs in the impregnation zone. After impregnation, the impregnated ligno-cellulosic feedstock is subjected to cooking. The wood chips are then cooked in huge pressurized vessels called digesters. Some digesters operate in batch manner and some in continuous processes. There are several variations of the cooking processes both for the batch and the continuous digesters. In a continuous digester the materials are fed at a rate which allows the pulping reaction to be complete by the time the materials exit the reactor. Typically delignification requires several hours at 170 to 176° C. Under these conditions lignin and hemicellulose degrade to give fragments that are soluble in the strongly basic liquid. The solid pulp (about 50% by weight based on the dry wood chips) is collected and washed. At this point the pulp is quite brown and is known as brown stock. The combined liquids, known as black liquor (so called because of its color), contain lignin fragments, carbohydrates from the breakdown of hemicellulose, sodium carbonate, sodium sulfate and other inorganic salts. One of the main chemical reactions that underpin the kraft process is the scission of ether bonds by the nucleophilic sulfide (S2−) or bisulfide (HS—) ions.

After cooking step, the excess black liquor is at about 15% solids and is concentrated in a multiple effect evaporator. After the first step the black liquor is about 20-30% solids. At this concentration the rosin soap rises to the surface and is skimmed off. The collected soap is further processed to tall oil. Removal of the soap improves the evaporation operation of the later effects.

The weak black liquor is further evaporated to reach a high dry matter which is typically between 65% to 80% (“heavy black liquor”) and burned in the recovery boiler to recover the inorganic chemicals for reuse in the pulping process. Higher solids in the concentrated black liquor increases the energy and chemical efficiency of the recovery cycle, but also gives higher viscosity and precipitation of solids (plugging and fouling of equipment). The molten salts (“smelt”) from the recovery boiler are dissolved in a process water known as “weak wash”. This process water, also known as “weak white liquor” is composed of all liquors used to wash lime mud and green liquor precipitates. The resulting solution of sodium carbonate and sodium sulfide is known as “green liquor”, although it is not known exactly what causes the liquor to be green. This liquid is mixed with calcium oxide, which becomes calcium hydroxide in solution, to regenerate the white liquor used in the pulping process. Calcium carbonate precipitates from the white liquor and is recovered and heated in a lime kiln where it is converted to calcium oxide (lime). Calcium oxide (lime) is reacted with water to regenerate the calcium hydroxide used. The combination of regeneration reactions forms a closed cycle with respect to sodium, sulfur and calcium and is the main concept of the so-called recausticizing process where sodium carbonate is reacted to regenerate sodium hydroxide.

The recovery boiler also generates high pressure steam which is fed to turbogenerators, reducing the steam pressure for the mill use and generating electricity. A modern kraft pulp mill is more than self-sufficient in its electrical generation and normally will provide a net flow of energy which can be used by an associated paper mill or sold to neighboring industries or communities through to the local electrical grid. Additionally, bark and wood residues are often burned in a separate power boiler to generate steam.

The finished cooked wood chips are blown by reducing the pressure to atmospheric pressure. This releases a lot of steam and volatiles. The steam produced can then be used to heat the pulp mill and any excess used in district heating schemes or to drive a steam turbine to generate electrical power. The volatiles are condensed and collected.

Screening of the pulp after pulping is a process wherein the pulp is separated from large shives, knots, dirt and other debris. The accept is the pulp. The material separated from the pulp is called reject.

The screening section consists of different types of sieves (screens) and centrifugal cleaning. The sieves are normally set up in a multistage cascade operation because considerable amounts of good fibres can go to the reject stream when trying to achieve maximum purity in the accept flow.

The fiber containing shives and knots are separated from the rest of the reject and reprocessed either in a refiner and/or is sent back to the digester. The content of knots is typically 0.5-3.0% of the digester output, while the shives content is about 0.1-1.0%.

The brown stock from the blowing goes to the washing stages where the used cooking liquors are separated from the cellulose fibers. Normally a pulp mill has 3-5 washing stages in series. Washing stages are also placed after oxygen delignification and between the bleaching stages as well. Pulp washers use counter current flow between the stages such that the pulp moves in the opposite direction to the flow of washing waters. Several processes are involved: thickening/dilution, displacement and diffusion. The dilution factor is the measure of the amount of water used in washing compared with the theoretical amount required to displace the liquor from the thickened pulp. Lower dilution factor reduces energy consumption, while higher dilution factor normally gives cleaner pulp. Thorough washing of the pulp reduces the chemical oxygen demand (COD). Several types of washing equipment are in use, such as for instance pressure diffusers, atmospheric diffusers, vacuum drum washers, drum displacers, wash presses.

Process chemicals may be added to improve the production process, such as: surfactants, which may be used to improve impregnation of the wood chips with the cooking liquors; anthraquinone which may be used as a digester additive; an emulsion breaker which can be added in the soap separation to speed up and improve the separation of soap from the used cooking liquors by flocculation; defoamers to remove foam and speed up the production process; dispersing, fixation detackifiers and complexing agents.

In another preferred embodiment, the process used to produce the cellulosic pulp comprises a sulfite pulping process, which may be modified to increase the removal of the hemicellulose from the ligno-cellulosic feedstock. Without limiting the way to implement the sulfite pulping process within the scope of the present disclosure, the sulfite pulping process may be described as follows. The sulfite process produces wood pulp which is almost pure cellulose fibers by using various salts of sulfurous acid to extract the lignin from wood chips in large pressure vessels called digesters. The salts used in the pulping process are preferably either sulfites (SO32−), or bisulfites (HSO3−), depending on the pH. The counter ion may comprise sodium (Na+), calcium (Ca2+), potassium (K+), magnesium (Mg2+) or ammonium (NH4+).

In the pulping preparation step, the pulping liquor for most sulfite mills is made by burning sulfur with the correct amount of oxygen to give sulfur dioxide, which is then absorbed into water to give sulfurous acid. Sulfuric acid is undesirable since it promotes hydrolysis of cellulose without contributing to delignification. The cooking liquor is prepared by adding the counter ions as hydroxides or carbonates. The relative amounts of each species present in the liquid depend largely on the relative amounts of sulfurous used.

In the pulping step, sulfite pulping is carried out between pH 1.5 and 5, depending on the counter ion to sulfite (bisulfite) and the ratio of base to sulfurous acid. The pulp is in contact with the pulping chemicals for 4 to 14 hours and at temperatures ranging from 130 to 160° C., again depending on the chemicals used.

Most of the intermediates involved in delignification in sulfite pulping are resonance-stabilized carbocations formed either by protonation of carbon-carbon double bonds or acidic cleavage of ether bonds which connect many of the constituents of lignin. It is the latter reaction which is responsible for most lignin degradation in the sulfite process. The electrophilic carbocations react with bisulfite ions (HSO3−) to give sulfonates.

The sulfite process does not degrade lignin to the same extent that the kraft process does and the lignosulfonates from the sulfite process are useful byproducts.

In the chemical recovery step, the spent cooking liquor from sulfite pulping is usually called brown liquor, but the terms red liquor, thick liquor and sulfite liquor are also used (compared to black liquor in the kraft process). Pulp washers, using countercurrent flow, remove the spent cooking chemicals and degraded lignin and hemicellulose. The extracted brown liquor is concentrated, in multiple effect evaporators. The concentrated brown liquor can be burned in the recovery boiler to generate steam and recover the inorganic chemicals for reuse in the pulping process or it can be neutralized to recover the useful byproducts of pulping. The sulfite process can use calcium, ammonium, magnesium or sodium as a base.

In another preferred embodiment, the process used to produce the cellulosic pulp comprises a soda pulping process, which eventually may be modified to increase the removal of the hemicellulose from the ligno-cellulosic feedstock. Soda pulping is a chemical process for making wood pulp with sodium hydroxide as the cooking chemical. In the Soda-AQ process, anthraquinone (AQ) may be used as a pulping additive to decrease the carbohydrate degradation. The soda process gives pulp with lower tear strength than other chemical pulping processes (sulfite process and kraft process), but has still limited use for easy pulped materials like straw and some hardwoods.

In an even more preferred embodiment, the process used to produce the cellulosic pulp comprises a dissolving pulp process. Dissolving pulp process comprises a pre-hydrolysis step to extract the most fraction of the hemicellulose before cooking, which is preferably performed according to the sulfite process or the kraft process. Pre-hydrolysis is conducted in the presence of water which may be in vapor (steam) or liquid phase, or a mixture of both phases. Pre-hydrolysis may be conducted in acid conditions by adding an external inorganic acid source or it can use ammonium, calcium, magnesium or sodium as a base. The pre-hydrolysis sulfate process produces pulp with a cellulose content up to 96%. Special alkaline purification treatments can yield even higher cellulose levels: up to 96 percent for the sulfite process and up to 98 percent for the sulfate process.

There are some variations in the type of hemicellulose found in different ligno-cellulosic feedstock, thereby the methods for hemicellulose extraction may differ slightly depending on wood species being processed at the plant under study. For instance, hardwoods have typically a high content of xylans while softwoods have a high content of glucomannans. For extraction of xylans from hardwoods, alkaline or near-neutral extraction is useful since xylans the xylans are extracted in their oligomeric form and can be further processed for use. For softwood hemicellulose like glucomannans, alkaline or near-neutral extraction cause rapid degradation of the polysaccharides and are not suitable extraction methods.

One of the key issue is to extract the hemicellulose without degradation of the polymeric sugars, or with a limited degradation, so that polymeric sugars can be used to produce high-value by products, such as for instance glycols comprising ethylene glycols and propylene glycols. For softwoods, extraction is preferably performed with water hydrolysis where most of the dissolved hemicellulose will be found in its oligomeric form in the hydrolyzate (the stream containing the extracted hemicellulose).

In the case that the process used to produce the cellulosic pulp comprises a chemical pulp treatment, it may be converted to a dissolving pulp process by adding a suitable pre-hydrolysis step. A preferred pre-hydrolysis step is described is in a following section of the present disclosure and comprises a soaking step of the ligno-cellulosic feedstock in the presence of a process fluid comprising water at a temperature between 100° C. and 2010° C. for a time between 1 minute to 24 hours, to produce a soaked liquid comprising solubilized oligomeric sugars and a soaked ligno-cellulosic feedstock, which may be further subjected to a steam explosion pulping process.

In another embodiment, the process used to produce the cellulosic pulp comprises an organosolv process Organosolv pulping involves contacting the ligno-cellulosic feedstock such as chipped wood with an aqueous organic solvent at temperatures preferably in the range of 140-220° C. This causes lignin to break down by hydrolytic cleavage of alpha aryl-ether links into fragments that are soluble in the solvent system. Solvents used include acetone, methanol, ethanol, butanol, ethylene glycol, formic acid, and acetic acid. The concentration of solvent in water is preferably in the range of 40% to 80%. Higher boiling solvents have the advantage of a lower process pressure. This is weighed against the more difficult solvent recovery by distillation. Ethanol has been suggested as the preferred solvent due to cost and easy recovery. Although butanol is shown to remove more lignin than other solvents and solvent recovery is simplified due to immiscibility in water, its high cost limits its use.

In another embodiment, the process used to produce the cellulosic pulp comprises a biological pulping, or biopulping process. In a biopulping process, certain species of fungi that are able to break down the unwanted lignin, but not the cellulose fibers. In the biopulping process, the fungal enzyme lignin peroxidase selectively digests lignin to leave remaining cellulose fibers. This could have major environmental benefits in reducing the pollution associated with chemical pulping. The pulp may be bleached using chlorine dioxide stage followed by neutralization and calcium hypochlorite. The oxidizing agent in either case oxidizes and destroys the dyes formed from the tannins of the wood and accentuated by sulfides present in it.

Preferred Hemicellulose Extraction Treatment

The liquid sugar stream is derived from the ligno-cellulosic feedstock by means of a treatment, or pre-treatment, of the ligno-cellulosic feedstock.

The pre-treatment of the ligno-cellulosic biomass is used to solubilize and remove carbohydrates, mainly xylans and glucans from the hemicellulose of the ligno-cellulosic feedstock, and at the same time the concentrations of harmful inhibitory by-products such as acetic acid, furfural and 5-hydroxymethylfurfural remain substantially low.

Pre-treatment techniques which may be used are well known in the art and include physical, chemical, and biological pre-treatments, or any combination thereof. In preferred embodiments the pre-treatment of ligno-cellulosic biomass is carried out as a batch or continuous process.

Physical pre-treatment techniques include various types of milling/comminution (reduction of particle size), irradiation

Comminution includes dry, wet and vibratory ball milling.

Although not needed or preferred, chemical pre-treatment techniques include acid, dilute acid, base, organic solvent, lime, ammonia, sulfur dioxide, carbon dioxide, pH-controlled hydrothermolysis, wet oxidation and solvent treatment.

If the chemical treatment process is an acid treatment process, it is more preferably, a continuous dilute or mild acid treatment, such as treatment with sulfuric acid, or another organic acid, such as acetic acid, citric acid, tartaric acid, succinic acid, or any mixture thereof. Other acids may also be used. Mild acid treatment means at least in the context of the invention that the treatment pH lies in the range from 1 to 5, preferably 1 to 3.

In a specific embodiment the acid concentration is in the range from 0.1 to 2.0% wt acid, preferably sulfuric acid. The acid is mixed or contacted with the ligno-cellulosic biomass and the mixture is held at a temperature in the range of around 160-220° C. for a period ranging from minutes to seconds. Specifically the pre-treatment conditions may be the following: 165-183° C., 3-12 minutes, 0.5-1.4% (w/w) acid concentration, 15-25, preferably around 20% (w/w) total solids concentration. Other contemplated methods are described in U.S. Pat. Nos. 4,880,473, 5,366,558, 5,188,673, 5,705,369 and 6,228,177.

Wet oxidation techniques involve the use of oxidizing agents, such as sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

In an embodiment both chemical and physical pre-treatment is carried out including, for example, both mild acid treatment and high temperature and pressure treatment. The chemical and physical treatment may be carried out sequentially or simultaneously.

The current strategies of thermal treatment are subjecting the ligno-cellulosic material to temperatures between 110-250° C. for 1-60 min e.g.:

Hot water extraction

Multistage dilute acid hydrolysis, which removes dissolved material before inhibitory substances are formed

Dilute acid hydrolysis at relatively low severity conditions

Alkaline wet oxidation

Steam explosion

Almost any pre-treatment with subsequent detoxification.

If a hydrothermal pre-treatment is chosen, the following conditions are preferred:

Pre-treatment temperature: 110-250° C., preferably 120-240° C., more preferably 130-230° C., more preferably 140-220° C., more preferably 150-210° C., more preferably 160-200° C., even more preferably 170-200° C. or most preferably 180-200° C.

Pre-treatment time: 1-60 min, preferably 2-55 min, more preferably 3-50min, more preferably 4-45 min, more preferably 5-40 min, more preferably 5-35 min, more preferably 5-30 min, more preferably 5-25 min, more preferably 5-20 min and most preferably 5-15 min.

Dry matter content after pre-treatment is preferably at least 20% (w/w). Other preferable higher limits are contemplated as the amount of biomass to water in the pre-treated ligno-cellulosic feedstock be in the ratio ranges of 1:4 to 9:1; 1.3.9 to 9:1, 1:3.5 to 9:1, 1:3.25 to 9:1, 1:3 to 9:1, 1:2.9 to 9:1, 1:2 to 9:1, 1.15 to 9:1, 1:1 to 9:1, and 1:0.9 to 9:1.

A preferred pretreatment of a ligno-cellulosic biomass include a soaking of the ligno-cellulosic biomass feedstock and optionally a steam explosion of at least a part of the soaked ligno-cellulosic biomass feedstock.

The soaking occurs in a substance such as water in either vapor form, steam, or liquid form or liquid and steam together, to produce a product. The product is a soaked biomass containing a soaking liquid, with the soaking liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

Either soaking step could also include the addition of other compounds, e.g. H2SO4, NH3, in order to achieve higher performance later on in the process.

In another embodiment, specifically in the case that a chemical process is used to produce the cellulosic pulp, such as for instance a kraft process or a sulfite process, the soaking step may include the addition of at least a portion of the chemical agent used in the chemical process for producing the cellulosic pulp.

The chemical agent is preferably selected from the group consisting of a sulfite, a bisulfite, sodium hydroxide and sodium carbonate. The sulfite and the bisulfite are salts whose counter ion may be sodium (Na+), calcium (Ca2+), potassium (K+), magnesium (Mg2+) or ammonium (NH4+).

In another embodiment, the soaking step may include a portion of the recycled chemical agents used in the chemical process. The recycled chemical agents may comprise a portion of the chemical agent which has been previously used, purified and recycled, or a derivative of the chemical agent, such as for instance the green liquor, the white liquor or the weak white liquor.

The product comprising the soaking liquid, or soaked liquid, is then passed to a separation step where at least a portion of the soaking liquid is separated from the soaked biomass. The liquid will not completely separate so that at least a portion of the soaking liquid is separated, with preferably as much soaking liquid as possible in an economic time frame. The liquid from this separation step is known as the soaked liquid stream comprising the soaking liquid. The soaked liquid will be the liquid used in the soaking, generally water and the soluble species of the feedstock. These water soluble species comprise glucan, xylan, galactan, arabinan, and their monomers and oligomers. The solid biomass is called the first solid stream as it contains most, if not all, of the solids.

The separation of the soaked liquid can again be done by known techniques and likely some which have yet been invented. A preferred piece of equipment is a press, as a press will generate a liquid under high pressure.

The first solid stream may then optionally be steam exploded to create a steam exploded stream, comprising solids. Steam explosion is a well-known technique in the biomass field and any of the systems available today and in the future are believed suitable for this step. The severity of the steam explosion is known in the literature as Ro, and is a function of time and temperature and is expressed as

Ro=t exp[(T−100)/14.75]

with temperature, T expressed in Celsius and time, t, expressed in minutes.

The formula is also expressed as Log(Ro), namely

Log(Ro)=Ln(t)+[(T−100)/14.75].

Log(Ro) is preferably in the ranges of 2.8 to 5.3, 3 to 5.3, 3 to 5.0 and 3 to 4.3.

The steam exploded stream may be optionally washed at least with water and there may be other additives used as well. It is conceivable that another liquid may be used in the future, so water is not believed to be absolutely essential. At this point, water is the preferred liquid. The liquid effluent from the optional wash may be added to the soaked liquid stream. This wash step is not considered essential and is optional.

The washed exploded stream is then processed to remove at least a portion of the liquid in the washed exploded material. This separation step is also optional. The term at least a portion is removed, is to remind one that while removal of as much liquid as possible is desirable (preferably by pressing), it is unlikely that 100% removal is possible. In any event, 100% removal of the water is not desirable since water is needed for the subsequent hydrolysis reaction. The preferred process for this step is again a press, but other known techniques and those not invented yet are believed to be suitable. The liquid products separated from this process may be added to the soaked liquid stream.

In an embodiment, the ligno-cellulosic biomass is exposed to a presoaking step before a soaking step in a temperature range of between 10° C. and 150° C., 25° C. to 150° C. even more preferable, with 25° C. to 145° C. even more preferable, and 25° C. to 100° C. and 25° C. to 90° C. also being preferred ranges.

The pre-soaking time could be lengthy, such as up to but preferably less than 48 hours, or less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

The pre-soaking step is done in the presence of a liquid which is the pre-soaked liquid. After soaking, this liquid preferably has removed less than 5% by weight of the total sugars in the raw material, more preferably, less than 2.5% by weight of the total sugars in the raw material being more preferable, with less than 1% by weight of the total sugars in the raw material, being the most preferred.

This pre-soaking step is useful as a modification to the soaking step of a biomass pre-treatment step. In soaking (not pre-soaking) of the biomass pre-treatment steps, the soaked liquid stream which has been separated from the soaked solids will preferably have reduced filter plugging components so that the soaked liquid can be easily purified.

The soaked liquid stream will comprise water, sugars which includes monomeric sugars and oligomeric sugars, salts which are dissociated into anions and cations in the soaked liquid stream, optionally phenols, furfural, oils and acetic acid. The soaked liquid stream may in particular contain xylooligomers and may further contain mannose and galactose and their respective oligomers.

Ideally, the concentration of the total sugars in the soaked liquid stream should be in the range of 0.1 to 300 g/l, with 50 to 290 g/l being most preferred, and 75 to 280 g/l even more preferred, with 100 to 250 g/l most preferred. This concentration can be done by the removal of water. A 50% removal of water increases the concentration of the non-water species by two. While various concentration increases are acceptable, in one embodiment, at least a two fold increase in the concentration of the hemicellulose-derived oligomeric sugars in the soaked liquid stream is reached. In one embodiment, at least a fourfold increase in the concentration of the hemicellulose-derived oligomeric sugars in the soaked liquid stream is reached. In one embodiment, at least a six fold increase in the concentration of the hemicellulose-derived oligomeric sugars in the soaked liquid stream is reached. Many concentration steps may be applied to the soaked liquid stream before or after each process step.

Production of the Liquid Stream Comprising Monomeric Sugars

The stream comprising hemicellulose-derived sugars may be subjected to a conditioning step comprising one or more purification steps. In the case that a chemical agent was used to solubilize the hemicellulose, the conditioning step removes at least a portion of the used chemical agent or derived compounds, preferably all or substantially all the used chemical agent or derived compounds.

Purification may be performed to remove the most fraction of the soluble and insoluble compounds which are not oligomeric and monomeric sugars and may be conducted by means of at least one technique selected from the group comprising chromatography, sedimentation, filtration, nanofiltration and ultrafiltration.

In a preferred embodiment, the soaking liquid stream, or the liquid stream comprising hemicellulose-derived oligomeric sugars, is subjected to hydrolysis for converting at least a portion of the oligomers to monomers. Hydrolysis of oligomers may be obtained by contacting the soaking liquid stream with a hydrolysis catalyst at hydrolysis conditions. The hydrolysis catalyst may be an inorganic acid, such as sulfuric acid, or an enzyme or enzyme cocktail. The hydrolysis conditions will vary according to the selected hydrolysis catalyst, and are well known in the art.

A preferred way to conduct the hydrolysis of the soaked liquid stream comprises at least two steps, according to the teaching of WO2013026849. The first step is to create an acidic stream from the soaked liquid stream. This is accomplished by increasing the amount of H+ ions to the soaked liquid stream to create the acidic stream. After the desired pH is obtained, the next step is hydrolyzing the oligosaccharides in the acidic stream by raising the temperature of the acidic stream to a hydrolysis temperature for the hydrolysis reaction to occur creating a hydrolyzed stream.

While the creation of the acidic stream can be done in any manner which increases the concentration of H+ ions, a preferred embodiment is to take advantage of the salt content of the soaked liquid stream. In order to obtain the required acidity for the hydrolysis step, the content of salts in the soaked liquid stream can be reduced via cation exchange while at the same time replacing the cations with H+ ions. While the salts may naturally occur in the soaked liquid stream, they can also be added as part of the pre-treatment processes or prior to or during the creation of the acidic stream.

In one embodiment, the hydrolyzed stream is a cleaner liquid, containing almost exclusively monomeric sugars, low content of salts and low amount of degradation products that could hinder subsequent chemical or biological transformations of the sugars.

In a preferred embodiment, the liquid sugar stream comprises at least a portion of the hydrolyzed stream.

In another preferred embodiment, the liquid sugar stream is comprised of at least a portion of the hydrolyzed stream.

Purification of the liquid stream may be performed before or after hydrolysis step. Purification may also include different steps, which may either preceding or following hydrolysis step.

Preferred Catalytic Conversion of the Liquid Sugar Stream to Polyols

The liquid sugar stream is converted to a mixture comprising polyols in a catalytic reaction involving an hydrogenation reaction of the monomeric sugars preferably in the presence of hydrogen.

In a preferred embodiment, the conversion process comprises two steps: a hydrogenation step of the liquid sugar stream to produce a hydrogenated mixture comprising water and at least a sugar alcohol; and a hydrogenolysis step of the hydrogenated mixture, to produce the polyols mixture.

The liquid sugar stream may be inserted in a hydrogenation reactor and contacted with a hydrogenation catalyst and hydrogen at hydrogenating conditions promoting the hydrogenation of the sugars in the liquid sugar stream. The hydrogenation catalyst is preferably a supported metal which comprises at least a metal selected from the group of Ru, Ni and Pt, or combination thereof. The catalyst support may comprise alumina, zirconia or activated carbon, or a combination thereof. The ratio between the total amount of the sugars in the liquid sugars stream to the amount of hydrogenation catalyst is preferably between 3:2 and 3:0.5.

Preferably, the hydrogenation reaction is conducted at a hydrogenation temperature promoting the conversion of all, or substantially all, the sugars in the liquid sugar stream. The hydrogenating temperature is between 50° C. to 200° C., preferably between 70° C. to 150° C., more preferably between 85° C. to 130° C., and most preferably between 100 to 120° C.

A process for converting a feedstock to a product or products may be run in a continuous or a batch operation. In batch operation, the process occurs in time-sequential steps in batches. A batch of feedstock is introduced into a reactor, then the conversion of the feedstock to the product or products takes place, then the product or products are removed from the reaction zone.

In a continuous process, the feedstock is being introduced as a stream into the reactor and a stream comprising the product or products is being removed, while the conversion is occurring. The streams may be introduced or removed continuously or discontinuously, being the process considered continuous in both cases.

The hydrogenation reaction may be conducted in a batch mode and for a hydrogenation time sufficient for converting all, or substantially all, the sugars in the liquid sugar stream. The hydrogenation time is preferably between 30 minutes to 240 minutes, more preferably between 45 minutes to 180 minutes, even more preferably between 60 minutes to 120 minutes. The catalyst is preferably present in particle form and dispersed in the liquid sugar stream to effectively promoting the hydrogenation reaction. The content of the hydrogenation reactor may be stirred during the reaction.

In another embodiment, the hydrogenation reaction is conducted in a continuous mode.

Preferably, the hydrogenation reactor is selected from the group consisting of fixed bed reactor, fluidized bed reactor and mechanically mixed reaction system. More preferably, the hydrogenation reactor is a fixed bed reactors, which may be operated as ebullating catalyst bed reactors or as a trickle bed reactors. In the most preferred embodiment, the reactor is a fixed bed operated as trickle bed reactors. Another type of reactor which may be used is the continuous stirred tank reactor (CSTR), which is a particular type of mechanically mixed reaction system, wherein the catalyst is dispersed in the liquid reagents and products. The continuous reaction time is conveniently expressed as the contact time between the liquid sugar stream and the hydrogenation catalyst, in the case that the hydrogenation reaction is conducted in a fixed bed configuration, or as residence time of the liquid sugar stream, in the case that the first reaction is conducted in a Continuous Stirred-Tank Reactor configuration. The continuous hydrogenation reaction time may be a value between 20 minutes and 3 h, preferably between 25 minutes and 2 h, and most preferably between 30 minutes and 1 hour.

The liquid sugar stream, the hydrogenation catalyst and the hydrogen may be introduced in the hydrogenation reactor separately from different inlets or may be premixed before the insertion in the reactor.

The hydrogenation reaction is conducted in the presence of hydrogen, at a hydrogenation pressure in the range of 30 bar to 150 bar, preferably in the range of 40 bar to 150 bar, more preferably in the range of 50 bar to 100 bar, most preferably in the range of 60 bar to 80 bar. In the case of hydrogenation conducted in a batch mode, the hydrogenation pressure corresponds to the pressure at which hydrogen is introduced in the reactor, at the temperature of 25° C. Experimentally, it is measured by a gauge pressure placed on the hydrogen gas supply line, close to the inlet of the hydrogenation reactor and immediately before sealing the reactor. The actual reaction pressure in the reactor may be different from the hydrogenation pressure due to the temperature effect and to contributions of gas reaction products and vapor pressure of the liquid sugar stream at the hydrogenation temperature. In the case of hydrogenation conducted in semi-continuous or continuous mode, wherein is the hydrogen gas flow to be regulated in order to control the pressure inside the reactor, the hydrogenation pressure is the actual pressure inside the reactor at the reaction temperature.

Preferably, the hydrogen and the liquid sugar stream are introduced in the hydrogenation reactor in suitable amounts to reach a molar ratio of the total amount of solubilized monomeric sugar to the hydrogen amount in a range of 1:2 to 1:10, more preferably of 1:3 to 1:8, and most preferably of 1:4: to 1:6. Because the reaction preferably occurs in an stoichiometric excess of Hydrogen for effectively promoting the hydrogenation reaction, a portion of the hydrogen will not react and may be recycled at the end of the reaction and reused in the whole conversion process. In the case of batch reaction, the total amount of hydrogen and the total amount of liquid sugar stream are introduced in the reactor which is then sealed. In the case of continuous or semi-continuous mode, the hydrogen and the liquid sugar stream are introduced in a continuous or semi-continuous way, preferably according the disclosed ranges.

In a preferred embodiment the liquid sugar stream comprises at least a compound selected from xylose, glucose and arabinose, or mixture thereof; depending on the ligno-cellulosic feedstock, it may further comprise mannose and galactose. The hydrogenation reaction of the sugars produces an hydrogenated mixture comprising at least a sugar alcohol. Preferred sugar alcohols are xylitol, sorbitol and arabitol, or mixture thereof.

In an even more preferred embodiment, the liquid sugar stream is derived from the ligno-cellulosic feedstock by solubilizing mainly the xylans of the ligno-cellulosic feedstock. Thereby, the sugars in the liquid sugar stream comprise mainly xylose and the preferred amount of xylose in the liquid sugar stream on a dry basis is greater than 50%, more preferably greater than 70%, even more preferably greater than 80%, yet even more preferably greater than 90%, being greater than 95% the most preferred value. The corresponding hydrogenated mixture will comprise mainly xylitol and the preferred amount of xylitol in the hydrogenated mixture on a dry basis is greater than 45%, more preferably greater than 70%, even more preferably greater than 80%, yet even more preferably greater than 90%, being greater than 95% the most preferred value.

The hydrogenated mixture is then removed from the hydrogenation reactor and inserted in the hydrogenolysis reactor. If the hydrogenation catalyst is present in dispersed particle form, it is at least in part removed from the reactor together with the hydrogenated mixture, and it may be recovered for instance by means of filtration and reinserted in the hydrogenation reactor, eventually after being regenerated. Eventually, also unwanted hydrogenation products may be removed from the hydrogenated mixture.

The hydrogenated mixture is inserted in a hydrogenolysis reactor and contacted with a hydrogenolysis catalyst and hydrogen at hydrogenolysis conditions promoting the hydrogenolysis of the sugar alcohols in the hydrogenated mixture. The hydrogenolysis catalyst comprises preferably a supported metal selected from the group of Ru, Ni and Pt, or combination thereof. The catalyst support may comprise alumina, zirconia or activated carbon, or a combination thereof. The ratio between the total amount of the alcohols in the hydrogenated mixture to the amount of hydrogenolysis catalyst is preferably between 3:2 and 3:0.1.

The hydrogenolysis reaction of the sugar alcohols occurs in the presence of OH— ions which affects the pH of the reaction environment. pH values greater than 9, corresponding to basic conditions, promote the effective hydrogenolysis of the sugar alcohols. OH— ions are preferably derived from a compound selected from the group consisting of NaOH, KOH, Ca(OH)2 and Ba(OH)2, or a combination thereof. The source of OH— ions may be introduced in the hydrogenolysis reactor or it may be added to the hydrogenated mixture before the insertion in the reactor.

The hydrogenolysis reaction is conducted at a hydrogenolysis temperature promoting the conversion of the alcohols in the hydrogenated mixture. The hydrogenolysis temperature may be a value between 150° C. to 240° C., and most preferably between 190 to 220° C.

The hydrogenolysis reaction may be conducted in a batch mode and for a hydrogenolysis time which is preferably sufficient for converting all, or substantially all, the sugar alcohols in the hydrogenated mixture. The hydrogenolysis time is preferably between 10 minutes to 10 hours, more preferably between 20 minutes to 8 hours, even more preferably between 30 minutes to 7 hours, yet even more preferably between 45 minutes to 6 hours, most preferably between 60 minutes to 4 hours, and even most preferably between 90 minutes to 3 hours. The hydrogenolysis catalyst is preferably present in particle form and dispersed in the hydrogenated mixture to effectively promoting the hydrogenolysis reaction. The content of the hydrogenolysis reactor may be stirred during the reaction.

In another embodiment, the hydrogenolysis reaction is conducted in a continuous mode. As in the case of hydrogenation reactor, the hydrogenolysis reactor may be selected from the group consisting of fixed bed reactor, fluidized bed reactor and mechanically mixed reaction system. More preferably, the hydrogenolysis reactor is a fixed bed reactors, which may be operated as ebullating catalyst bed reactors or as a trickle bed reactors. In the most preferred embodiment, the reactor is a fixed bed operated as trickle bed reactors. The reaction time is conveniently expressed as the contact time between the hydrogenated mixture and the hydrogenolysis catalyst, in the case that the hydrogenolysis reaction is conducted in a fixed bed configuration, or as residence time of the hydrogenated mixture, in the case that the hydrogenolysis reaction is conducted in a Continuous Stirred-Tank Reactor configuration. The continuous hydrogenolysis reaction time may be a value between 15 minutes and 10 h, preferably between 20 minutes and 5 h, more preferably between 25 minutes and 2 hour, and most preferably between 30 minutes and 1 h.

The hydrogenolysis reaction is conducted in the presence of hydrogen of a hydrogen, at a hydrogenolysis pressure in the range of 40 bar to 170 bar, preferably in the range of 40 bar to 150 bar, more preferably in the range of 50 bar to 100 bar, most preferably in the range of 60 bar to 80 bar. In the case of hydrogenolysis conducted in a batch mode, the hydrogenolysis pressure corresponds to the pressure at which hydrogen is introduced in the reactor, at the temperature of 25° C. Experimentally, it is measured by a gauge pressure placed on the hydrogen gas supply line, close to the inlet of the hydrogenolysis reactor and immediately before sealing the reactor. The actual reaction pressure in the reactor may be different from the hydrogenolysis pressure due to the temperature effect and to contributions of gas reaction products and vapor pressure of the liquids at the hydrogenolysis temperature. In the case of hydrogenolysis conducted in continuous mode, wherein is the hydrogen gas flow to be regulated in order to control the pressure inside the reactor, the hydrogenolysis pressure is the actual pressure inside the reactor at the reaction temperature

Preferably, the hydrogen and the hydrogenated mixture are introduced in the hydrogenolysis reactor in suitable amounts to have a molar ratio of the total amount of sugar alcohols to the hydrogen amount in a range of 1:2 to 1:10, more preferably of 1:3 to 1:8, and most preferably of 1:4: to 1:6. Because the reaction preferably occurs in a stoichiometric excess of Hydrogen for effectively promoting the hydrogenolysis reaction, a portion of the hydrogen will not react and may be recycled at the end of the reaction and reused in the whole conversion process. In the case of batch reaction, the total amount of hydrogen and the total amount of hydrogenated mixture are introduced in the reactor which is then sealed. In the case of continuous mode, the hydrogen and the hydrogenated mixture are introduced in a continuous or semi-continuous way, preferably according the disclosed ranges.

The hydrogenolysis reaction of the sugar alcohols in the hydrogenated mixture produces a polyols mixture comprising water, primary polyols and secondary polyols.

The primary polyols may comprise at least a compound selected from the group consisting of ethylene glycol, propylene glycol, and mixture thereof.

The secondary polyols may comprise at least a compound selected from the group consisting of glycerol, arabitol, threitol, erythrithol and an unreacted sugar alcohol, and mixture thereof.

The polyols mixture may further comprise other polyols, unwanted compounds, comprising lactic acid, glycolaldehyde or formic acid, and unreacted sugars and sugar alcohols.

The polyols mixture is then removed from the hydrogenolysis reactor. If the hydrogenolysis catalyst is present in dispersed particle form, it is at least in part removed from the reactor together with the polyols mixture, and it may be recovered for instance by means of filtration and reinserted in the hydrogenolysis reactor, eventually after being regenerated.

The polyols stream comprising primary polyols may be recovered from the polyols mixture by any process known in the art and still to be invented.

In a preferred embodiment, a portion of the water of the polyols mixture is first removed by means of a dewatering step. Dewatering may be done by thermal evaporation or by filtration. Preferably, the dry matter content of the dewatered glycols mixture is a value in the range of 40% to 95%, more preferably of 50 to 90% even more preferably of 60% to 85%, and most preferably of 70% to 80%.

The polyols mixture, which has been eventually dewatered, is then separated into at least the polyols stream comprising ethylene glycol and/or propylene glycol, and at least a residual stream, comprising water and the at least a secondary polyols which is not ethylene glycol and propylene glycol, which is preferably glycerol. Depending on the separation conditions, the polyols stream may further comprise other polyols, such as for instance butanediol, pentanediols, and/or other compounds which are not polyols, such as for instance unreacted compounds, intermediates or byproducts. The residual stream may further comprise other polyols, such as arabitol, threitol, erythrithol, and unreacted sugar alcohols, which are not separated at the separation conditions and/or other compounds which are not polyols, such as for instance lactic acid, formic acid (which in a basic environment may be present as a salt, such as for instance sodium lactate and sodium formate), glycolaldehyde. Depending on the separation conditions, the residual stream may further comprise a portion of ethylene glycol and propylene glycol of the polyols mixture. Other additional residual streams may be produced in the separation step.

The preferred way for separating the polyols mixture is by thermal evaporation, which may be conducted at a temperature between 100° C. and 140° C. and at a pressure between 30 mbar and 200 mbar, more preferably at a temperature of about 120° C. and at a pressure of about 50 mbar.

An ethylene glycol stream and a propylene glycol stream may be separated from the polyols mixture by means of any process known in the art and still to be invented, preferably by means of distillation. Optionally, other additional residual streams are produced in the separation.

The propylene glycol stream comprises propylene glycol, and may further comprise small amount of ethylene glycol or other polyols.

The ethylene glycol stream comprises a plurality of diols, wherein ethylene glycol is the main component, as the amount of ethylene glycol, expressed as molar percent with respect to the plurality of diols, is preferably greater than 80%. In preferred embodiments, the molar amount of ethylene glycol is greater than 85%, being greater than 90% more preferable, greater than 95% even more preferable and greater than 98% the most preferable value.

In an embodiment, the ethylene glycol stream further comprises at least one diol selected from 1,2-Propylene glycol, 1,2-Butanediol and 1,2-Pentanediol.

In a preferred embodiment, the ethylene glycol stream comprises 1,2 Propylene glycol, and the percent molar amount of 1,2 Propylene glycol with respect to the plurality of diols is preferably less than 15%, more preferably less than 12%, even more preferably less than 10%, even yet more preferable less than 7%, even yet more preferable less than 5%, most preferably less than 3%, being less than 2% the even most preferred value.

In another preferred embodiment, the ethylene glycol stream comprises 1,2-Butanediol, and the percent amount of 1,2-Butanediol with respect to the plurality of diols is preferably less than 10%, more preferably less than 8%, even more preferably less than 5%, even yet more preferable less than 3%, most preferably less than 2%, being less than 1% the even most preferred value.

In a preferred embodiment, the ethylene glycol stream comprises 1,2-Pentanediol, and the percent amount of 1,2-Pentanediol with respect to the plurality of diols is preferably less than 5%, more preferably less than 4%, even more preferably less than 3%, even yet more preferable less than 2% and most preferably less than 1%.

Even if the ethylene glycol stream may comprise only one 1,2-diol, more preferably it comprises two 1,2-diols, even more preferably it comprises three 1,2-diols. Most preferably, the ethylene glycol stream comprises 1,2-Propylene glycol, 1,2-Butanediol and 1,2-Pentanediol.

The ethylene glycol stream may be used to produce a polyester resin.

A first preferred method to produce the polyester resin is the ester process, comprising an ester interchange and a polycondensation. Basically, the diols of the plurality of diols are reacted with a dicarboxylic ester (such as dimethyl terephthalate) in an ester interchange reaction, which may be catalyzed by an ester interchange catalyst. As an alcohol is formed in the reaction (methanol when dimethyl terephthalate is employed), it may be necessary to remove the alcohol to convert all or almost all of the reagents into monomers. Then monomers undergo polycondensation and the catalyst employed in this reaction is generally an antimony, germanium or titanium compound, or a mixture thereof. The ester interchange catalyst may be sequestered to prevent yellowness from occurring in the polymer by introducing a phosphorus compound, for example polyphosphoric acid, at the end of the ester interchange reaction.

A second preferred method to produce the polyester resin is the acid process, comprising a direct esterification and a polycondensation. Basically, the diols of the plurality of diols are reacted with an acid (such as terephthalic acid) by a direct esterification reaction producing monomer and water, which is removed to drive the reaction to completion. The direct esterification step does not require a catalyst. Similarly to the ester process, the monomers then undergo polycondensation to form polyester.

In both method, the polyester may be further polymerized to a higher molecular weight by a solid state polymerization, which is particularly useful for container (bottle) application.

In a preferred embodiment, at least 85% of the acid moieties of the polyester are derived from terephthalic acid or its dimethyl ester.

Thermal Energy Recovery from the Residual Stream

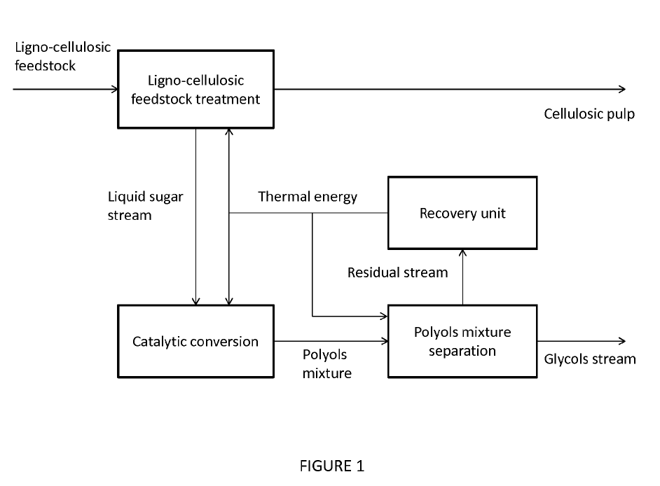

In the disclosed process, the residual stream generated in the polyols mixture separation is used to produce a thermal energy in a recovery unit, as depicted in FIG. 1. The residual stream has a dry matter content which may be greater than 80%, more preferably greater than 85%, even more preferably greater than 90%, even yet more preferably greater than 93%, even yet more preferably greater than 95%, and most preferably greater than 98%. Being the percent water content of the residual stream significantly less than the hemicellulose-derived sugar stream, the recovery of thermal energy from the residual stream, with respect to the recovery of thermal energy from the hemicellulose-derived sugar stream, has the advantage that less energy has to be spend in the evaporation of water.

In a preferred embodiment, wherein the polyols separation occurs by means of a thermal process such us distillation or evaporation, the residual stream is separated at a temperature at which it is characterized by a low viscosity, thereby being possible to pump it through pipes. Preferably, after separation, the residual stream is maintained at a temperature preserving a flow capability of the residual stream and transported to the recovery unit by means of a pipe or pipes. The residual stream is inserted into the recovery unit at a temperature which is preferably greater than 60° C., more preferably greater than 70° C., and most preferably greater than 80° C. Optionally, the water content of the residual stream may be further reduced, by means of filtration or thermal evaporation techniques.

The recovery unit may comprise any mean for recovering thermal energy from the residual stream. In a preferred embodiment, thermal energy is recovered by burning the residual stream, which may occur preferably in combustion conditions, that is in presence of oxygen, even if pyrolysis conditions corresponding to a depletion or absence of oxygen may also be used, depending on the properties and composition of the residual stream. The recovery unit may comprise a combustion chamber, wherein the residual stream is inserted preferably in droplets form to enhance combustion process. In a preferred embodiment, thermal energy is used to produce steam in a steam generator, which may be adapted for burning the residual stream.

The thermal energy, preferably in the form of steam, may then be used to supply at least a portion of the thermal energy demand of the disclosed process. The thermal energy recovered from the residual stream may be used in any of the steps of the disclosed process, in particular for treating the ligno-cellulosic feedstock to produce the liquid sugar stream and the cellulosic pulp, and/or in the conversion of the liquid sugar stream to the polyols mixture, and/or in the polyols mixture separation. In an embodiment, the recovery unit comprises a cogeneration system, wherein the residual stream is burnt to generate steam which is used to produce electric energy for supplying at least a portion of the electric energy demand of the disclosed process. Optionally, at least a portion of the produced electric energy is supplied to an external electric network.

In another embodiment, the recovery unit comprises means for converting the residual stream to an intermediate compound, which is then used to recover the thermal energy. Preferably, the intermediate compound is a burnable gas or gas mixture, such as hydrogen and methane, which may be obtained for instance by means of a process comprising a gasification step of the residual stream or by means of a biological process, such as aerobic or anaerobic fermentation. The burnable gas or gas mixture is then used as fuel for producing thermal energy and optionally electric energy.

In the case that the process used to produce the cellulosic pulp and the liquid sugar stream comprises a chemical pulp treatment in the presence of a chemical agent, such as for instance a kraft process or a sulfite process, at least an effluent stream is also produced. The at least an effluent stream comprises at least a portion of the lignin of the ligno-cellulosic feedstock, which may be in soluble or insoluble form, and at least a portion of the chemical agent, which at least in part may have been reacted to produce a derivative of the chemical agent. The at least an effluent stream may optionally comprise a portion of the carbohydrates of the ligno-cellulosic feedstock which has been removed in the chemical process and may be in soluble or insoluble form. The at least an effluent stream is sent to a recovery unit which is known in the art of pulp industry as recovery boiler, wherein it is converted to thermal energy and at least a portion of the chemical agent or a derivative compound of the chemical agent are recovered and for being reused in the treatment of the ligno-cellulosic feedstock. Depending on the chemical process used, in the pulp industry the at least an effluent stream may be indicated in the art for instance as black liquor or brown liquor, and may have different composition. The stream comprising the recycled chemical agent or the derivative compound or compounds of the chemical agent may be indicated as green liquor or white liquor. Different configurations of recovery boiler, developed so far and still to be invented, may be used in the disclosed process. In a preferred configuration, the organic portion of chemicals produces heat by combustion which is used to produce high pressure steam, which is used to generate electricity in a turbine. The turbine exhaust, low pressure steam is used for process heating.

In a most preferred embodiment, the thermal energy is recovered from the residual stream comprising the majority by weight of the total amount of the secondary polyols and the at least an effluent stream in a unique recovery unit. Preferably, the recovery unit is a recovery boiler used in the pulp industry, which may be adapted for being fed from the two streams. The two streams may be inserted in two different combustion chambers in the recovery boiler, wherein they are burnt separately to generate steam. Preferably, the recovery boiler is adapted to burn the residual stream and the at least an effluent stream in a unique combustion chamber. In this case, the two streams may be inserted from two separated inlets, or may be premixed to form a unique stream before being inserted in the unique combustion chamber.

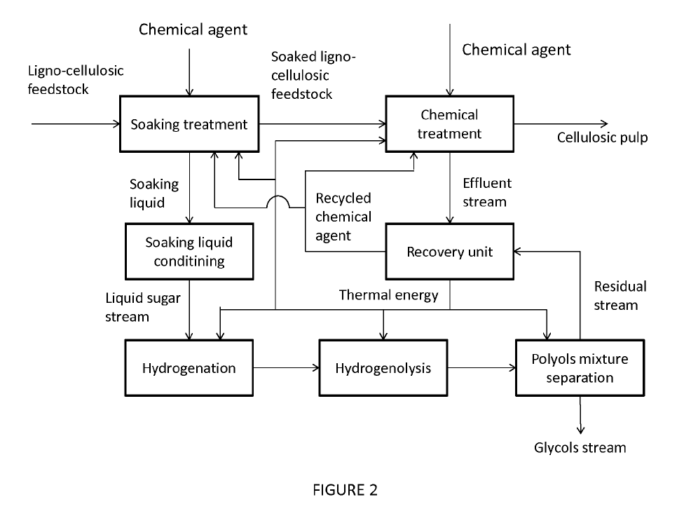

As depicted in FIG. 2, which schematically represents an embodiment of the disclosed process in a detailed way, the thermal energy may be used in the soaking step of the ligno-cellulosic biomass, the chemical treatment of the soaked ligno-cellulosic biomass, the hydrogenation, hydrogenolysis step, or the polyols mixture separation.

Optionally, apart from the residual stream or streams comprising the majority by weight of the total amount of the secondary polyols, additional residual streams comprising organic compounds may be created in many steps of the disclosed process and may be used to recover thermal energy. The additional streams may be concentrated to reduce the water content according to the technical requirements of the recovery unit, in particular of the recovery boiler. Preferably, the concentration of chloride in the additional residual streams is less than 2 g/l, more preferably less than 1.5 g/l, even more preferably less than 1.0 g/l, even yet more preferably less than 0.5 g/l, and most preferably less than 0.1 g/l. In the most preferred embodiment, the additional residual streams do not contain or substantially do not contain chloride. Chloride may be present in elemental form or in chloride compounds. As examples of additional residual streams, a first additional residual stream may be produced by separation of at least a portion of unreacted monomeric sugars and/or unwanted organic compounds from the hydrogenated mixture produced in the hydrogenation step of the catalytic conversion of the liquid sugar stream. A second additional residual stream may be produced from the conditioning of the soaking liquid, wherein the purification steps may generate a stream comprising oligomeric sugars which have not been hydrolyzed to monomeric sugars.

Feedstock Selection

In general, a natural or naturally occurring ligno-cellulosic biomass can be one feed stock for this process. Ligno-cellulosic materials can be described as follows:

Apart from starch, the three major constituents in plant biomass are cellulose, hemicellulose and lignin, which are commonly referred to by the generic term lignocellulose. Polysaccharide-containing biomasses as a generic term include both starch and ligno-cellulosic biomasses. Therefore, some types of feedstocks can be plant biomass, polysaccharide containing biomass, and ligno-cellulosic biomass.

Polysaccharide-containing biomasses according to the present invention include any material containing polymeric sugars e.g. in the form of starch as well as refined starch, cellulose and hemicellulose.

Relevant types of naturally occurring biomasses for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all because the characterization is primarily to the unique characteristics of the lignin and surface area.

The ligno-cellulosic biomass feedstock used to derive the composition is preferably from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins usually entire. The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glumes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indian grass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

Another naturally occurring ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants. The two main groups in which secondary xylem can be found are:

1) conifers (Coniferae): there are some six hundred species of conifers. All species have secondary xylem, which is relatively uniform in structure throughout this group. Many conifers become tall trees: the secondary xylem of such trees is marketed as softwood.

2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood useful in this process is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood useful for this process is used to describe wood from trees that belong to the angiosperm family. Angiosperms are plants with ovules enclosed for protection in an ovary. When fertilized, these ovules develop into seeds. The hardwood trees are usually broad-leaved; in temperate and boreal latitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore a preferred naturally occurring ligno-cellulosic biomass may be selected from the group consisting of the grasses and woods. Another preferred naturally occurring ligno-cellulosic biomass can be selected from the group consisting of the plants belonging to the conifers, angiosperms, Poaceae and families. Another preferred naturally occurring ligno-cellulosic biomass may be that biomass having at least 10% by weight of it dry matter as cellulose, or more preferably at least 5% by weight of its dry matter as cellulose.

The carbohydrate(s) comprising the invention is selected from the group of carbohydrates based upon the glucose, xylose, and mannose monomers and mixtures thereof.

1-17. (canceled)

18. An integrated process for producing at least a cellulosic pulp comprising cellulose in the form of fibers and a polyols stream from a ligno-cellulosic feedstock comprising cellulose, hemicellulose and lignin, wherein the process comprises the steps of:
a) treating the ligno-cellulosic feedstock to produce the cellulosic pulp and at least a liquid sugar stream comprising water and monomeric sugars derived from the hemicellulose of the lignocellulosic feedstock;
b) catalytically converting the monomeric sugars of the liquid sugar stream to a polyols mixture, comprising primary polyols and secondary polyols;
c) separating at least a portion of the polyols mixture to produce at least the polyols stream and a residual stream, wherein the polyols stream comprises the majority by weight of the primary polyols and the residual stream comprises the majority by weight of the secondary polyols;
d) recovering a first thermal energy from the residual stream in a first recovery unit.

19. The integrated process of claim 18, wherein the treatment of the ligno-cellulosic feedstock comprises the steps of:
i. soaking the ligno-cellulosic feedstock in the presence of a process fluid comprising water at a temperature between 100° C. and 210° C. for a time between 1 minute and 24 hours to produce a soaking liquid comprising oligomeric sugars and a soaked ligno-cellulosic feedstock;
ii. treating the soaking liquid comprising oligomeric sugars to produce at least the liquid stream comprising monomeric sugars;
iii. treating the soaked ligno-cellulosic feedstock in the presence of a chemical agent to produce at least a cellulosic stream comprising the cellulosic pulp and an effluent stream comprising at least a portion of the lignin and at least a portion of the chemical agent or a derivative of the chemical agent;
iv. recovering the cellulosic pulp, and
v. recovering from the effluent stream a recycled chemical agent comprising the chemical agent or the derivative of the chemical agent and a second thermal energy in a second recovery unit.

20. The integrated process of claim 18, wherein the process fluid further comprises a portion of the chemical agent or a portion of the recycled chemical agent.

21. The integrated process of claim 19, wherein the chemical agent comprises at least a compound selected from the group consisting of a sulfite, a bisulfite, sodium hydroxide and sodium carbonate.

22. The integrated process of claim 21, wherein the sulfite comprises a counter ion which is selected from the group consisting of sodium ion, calcium ion, potassium ion, magnesium ion, and ammonium ion.

23. The integrated process of claim 22, wherein the bisulfite comprises a counter ion which is selected from the group consisting of sodium ion, calcium ion, potassium ion, magnesium ion, and ammonium ion.

24. The integrated process of claim 18, wherein the catalytic conversion comprises the steps of:
a) hydrogenating the liquid sugar stream by contacting the liquid sugar stream with a hydrogenation catalyst in the presence of Hydrogen, at a hydrogenation pressure in the range of 30 bar to 150 bar and at a hydrogenation temperature in the range of 50° C. to 200° C., and for a hydrogenation time sufficient to produce a hydrogenated mixture comprising water and at least a sugar alcohol;
b) conducting hydrogenolysis of at least a portion of the hydrogenated mixture, by contacting the at least a portion of the hydrogenated mixture with a hydrogenolysis catalyst int eh presence of OH′ ions and Hydrogen, at a hydrogenolysis pressure in the range of 40 bar to 170 bar, at a hydrogenolysis temperature and for a hydrogenolysis time sufficient to produce the polyols mixture.

25. The integrated process of claim 19, wherein the catalytic conversion comprises the steps of:
a) hydrogenating the liquid sugar stream by contacting the liquid sugar stream with a hydrogenation catalyst in the presence of Hydrogen, at a hydrogenation pressure in the range of 30 bar to 150 bar and at a hydrogenation temperature in the range of 50° C. to 200° C., and for a hydrogenation time sufficient to produce a hydrogenated mixture comprising water and at least a sugar alcohol;
b) conducting hydrogenolysis of at least a portion of the hydrogenated mixture, by contacting the at least a portion of the hydrogenated mixture with a hydrogenolysis catalyst int eh presence of OH′ ions and Hydrogen, at a hydrogenolysis pressure in the range of 40 bar to 170 bar, at a hydrogenolysis temperature and for a hydrogenolysis time sufficient to produce the polyols mixture.

26. The integrated process of claim 18, wherein the primary polyols comprise at least a compound selected from the group consisting of ethylene glycol, propylene glycol, and mixture thereof.

27. The integrated process of claim 24, wherein the secondary polyols comprise at least a compound selected from the group consisting of glycerol, arabitol, threitol, erythrithol and an unreacted sugar alcohol, and mixture thereof.

28. The integrated process of claim 25, wherein the secondary polyols comprise at least a compound selected from the group consisting of glycerol, arabitol, threitol, erythrithol and an unreacted sugar alcohol, and mixture thereof.

29. The integrated process of claim 24, wherein the residual stream further comprises a compound selected from the list consisting of lactic acid, formic acid, and glycolaldehyde.

30. The integrated process of claim 25, wherein the residual stream further comprises a compound selected from the list consisting of lactic acid, formic acid, and glycolaldehyde.

31. The integrated process of claim 18, wherein the residual stream has a percent dry matter by weight which is a value greater than 80, 85, 90, 93, 95, and 98.

32. The integrated process of claim 18, wherein the residual stream is inserted in the first recovery unit at a temperature which is greater than a value selected from the group consisting of 60° C., 70° C., and 80° C.

33. The integrated process of claim 19, wherein the first recovery unit and the second recovery unit are the same recovery unit.

34. The integrated process of claim 33, wherein at least a portion of the residual stream and at least a portion of the effluent stream are combined together before being inserted in the recovery unit.

35. The integrated process of claim 33, wherein the recovery unit comprises a recovery boiler.

36. The integrated process of claim 18, wherein at least a portion of the first thermal energy and/or at least a portion of the second thermal energy are used in the integrated process.

37. The integrated process of claim 18, wherein at least a portion of the first thermal energy and/or at least a portion of the second thermal energy are converted to electrical energy.
